(19) United States
(12) Patent Application Publication
(10) Pub. No.: US 2025/0213107 A1
(43) Pub. Date: Jul. 3, 2025

(54) Lighted Dental Mirror

(71) Applicant: DESIGNS FOR VISION, INC., Bohemia, NY (US)

(72) Inventors: Richard E. Feinbloom, New York, NY (US); Moty Solomon, Beit uziel (IL)

(21) Appl. No.: 18/850,529

(22) PCT Filed: May 16, 2023
(86) PCT No.: PCT/US23/22372

§ 371 Date: Sep. 25, 2024

Related U.S. Application Data
(60) Provisional application No. 63/343,047, filed on May 17, 2022.

(51) Int. Cl. A61B 1/247; A61B 1/06

(52) U.S. Cl. CPC A61B 1/247; A61B 1/0638

(57) ABSTRACT

An induction-based lighting system comprising coil assembly, and a lighted LED assembly incorporated onto existing dental mirror devices that provides light to areas viewed by the mirror element when the lighted LED assembly is inserted within or placed within a proximity to the coil assembly.

### CLAIM OF PRIORITY

This applicant claims, pursuant to 35 USC 371, as a National Stage filing, priority to and the benefit of the filing date of that patent application filed under the terms of the Patent Cooperation Treaty afforded serial number PCT/US2023/22372 filed on May 15,2023, which claimed, pursuant to 35 USC 119, priority to and the benefit of the earlier filing date of that patent application filing in the US Patent and Trademark Office on May 17, 2022 and afforded Ser. No. 63/343,047, the contents of all of which are incorporated by reference, herein.

### BACKGROUND OF THE INVENTION

### Background

Generally, medical and dental practitioners utilize overhead lighting to direct light into the body cavity to increase the intensity of the light projected into a body cavity in order to view areas within the body cavity. Generally, the overhead lighting is halogen in nature, but Light Emitting Diode (LED) technology has also been introduced in overhead lighting to provide the practitioner with a means to increase the viewability of areas within the body cavity. In other aspect, LED lighting has been used to apply emitted light to a specific area within the body cavity. LED technology has also allowed the practitioner to utilize light of different wavelengths (or colors) to distinguish healthy tissue from diseased tissue.

In many cases, medical and/or dental practitioners utilize mirrors to view areas of the body cavity that would otherwise not be visible to the practitioner. For example, dentists are known to use mirrors to view the regions within the oral cavity that would otherwise not be vision without the dentist contouring their body into an awkward configuration.

However, dental mirrors have generally been limited to a mirror extending from a handle, wherein the mirror is rotated and/or maneuvered within the oral cavity to enable light from the overhead lighting to be projected onto a desired region.

Hence, there is a need in the industry for a lighted mirror configuration that may be utilized by practitioners to provide light directly to areas that are to be viewed by the practitioner.

### SUMMARY OF THE INVENTION

In one aspect of the invention, a lighted mirror system is disclosed that directs a light directly toward an area that may be viewed through a mirror.

In another aspect of the invention, a lighted mirror system is disclosed that provides light of one or more known wavelengths directly toward an area that may be viewed through a mirror.

In another aspect of the invention, a self-powered lighted mirror system is disclosed that provides light of one or more known wavelengths directly toward an area that may be viewed through a mirror.

In another aspect of the invention, a system for providing a light directly toward an area being viewed through a mirror is disclosed.

### BRIEF DESCRIPTION OF THE FIGURES

The advantages, nature, and various additional features of the invention will appear more fully upon consideration of the illustrative embodiments described in detail in connection with the accompanying drawings, where like or similar reference numerals are used to identify like or similar elements throughout the drawings:

FIG. 1 illustrates a front view of a first exemplary embodiment of a lighted LED assembly in accordance with the principles of the invention.

FIG. 2 illustrates an exploded prospective side view of the lighted LED assembly shown in FIG. 1.

FIG. 3 illustrates a prospective side view of a second exemplary embodiment of a lighted LED assembly in accordance with the principles of the invention.

FIG. 4 illustrates a side view of a third exemplary embodiment of a lighted LED assembly in accordance with the principles of the invention.

FIG. 5 illustrates a prospective view of a lighting system utilizing the LED assemblies disclosed, herein.

FIG. 6 illustrates a block diagram of a first exemplary embodiment of the lighted LED assembly shown in FIG. 5.

FIG. 7 illustrates a circuit diagram of a second exemplary embodiment of the lighted LED assembly shown in FIG. 5.

FIG. 8 illustrates a circuit diagram of an exemplary circuit configuration suitable for powering the LEDs shown in FIG. 5.

FIG. 9 illustrates a block diagram of an exemplary circuit configuration suitable for powering the coil shown in FIG. 5.

It is to be understood that the figures, which are not drawn to scale, and descriptions of the present invention described herein have been simplified to illustrate the elements that are relevant for a clear understanding of the present invention, while eliminating, for purposes of clarity, many other elements. However, because these omitted elements are well-known in the art, and because they do not facilitate a better understanding of the present invention, a discussion of such elements are not provided herein. The disclosure, herein, is directed also to variations and modifications known to those skilled in the art.

### DETAILED DESCRIPTION OF THE INVENTION

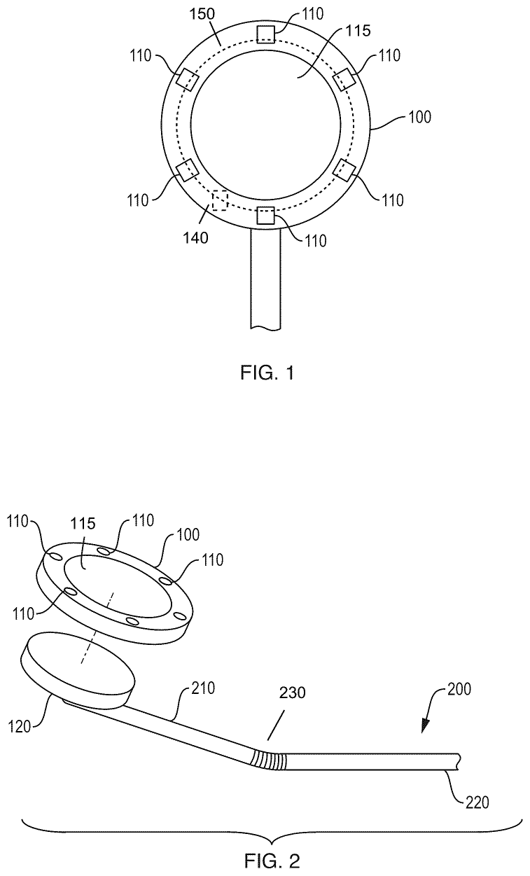

FIG. 1 illustrates a first exemplary embodiment of a lighted LED assembly in accordance with the principles of the invention.

In this illustrated embodiment lighted LED assembly 100, shown in the form of ring, comprises a plurality of light emitting diode (LED) elements 110 distributed substantially uniformly about an open center area 115. Although lighted LED assembly 100 is shown in the form of a circular ring (i.e., open center), it would be recognized that lighted LED assembly 100 may similarly be in an elongated or rectangular form with open center without alternating the scope of the invention claimed. Light emitting diodes elements 110 may be composed of one or more lighting sources configurated to emit light in one or more wavelength ranges. For example, light elements 110 may be composed of lighting sources that emit light in a visible wavelength range. For example, a white wavelength band (e.g., 380-700 nanometers (nm)) or in one or more of a blue wavelength band (e.g., 400-465 nm), a cyan wavelength band, a green wavelength band, a yellow wavelength band, an orange wavelength band and/or a red wavelength band. Alternatively, lighting elements 110 may emit light in an ultra-violet wavelength band (10-380 nm) and/or an infra-red wavelength band (greater than 700 nm); or any combination of visible, ultra-violet and infra-red wavelength bands. In one aspect of the invention, lighting elements 110 may emit light at one or more wavelengths within a same wavelength band. For example, selected ones of lighting elements 110 may emit light in a lower range of the blue wavelength band whereas selected other ones of lighting elements 110 may separately or concurrently emit light in an upper range of the blue wavelength band. In another aspect of the invention, selected ones of the plurality of light emitting diodes 110 may concurrently emit white light and/or one or more blue light wavelengths, wherein the concurrent emission of white light may be at a lower intensity when emitted with blue light than when the white light is emitted by itself.

In accordance with the principles of the invention, lighted LED assembly 100 may be composed of an optically clear material (e.g., silicon) that allows light emitted by lighting elements 110 to pass through LED assembly 100 without alteration and/or attenuation.

Incorporated within LED assembly 100 is electrical circuity 140 and coil 150, wherein electrical circuitry 140 comprises well-known electrical and electronic elements (e.g., resistors, capacitors, diodes, transistor, integrated circuitry (not shown)), that may be used to control the flow of electrical energy (voltage, current) to one or more of lighting elements 110. Coil 150, as will be discussed, operates as an inductive coil to provide an induced electrical energy (i.e., voltage) to circuitry 140. Circuitry 140 receiving the induced electrical energy comprises elements to convert the induced electrical energy to a substantially steady state voltage/current (i.e., power) that may then be used to power lighting elements (LEDs) 110.

FIG. 2 illustrates an exploded prospective side view of lighted LED assembly 100 incorporated onto a mirror (i.e., highly reflective) element 120 of assembly 200, wherein highly reflective element 120 is extended from handle element 220 by extension arm 210.

In accordance with the principles of the invention, lighted LED assembly 100 may be attached to mirror element 120 through a snap fit connection, a form fit connection, a screw connection, a bayonet connection, etc., wherein mirror element 120 extends through, or is viewing through, opening 115. Alternatively, LED assembly 100 may include a slot (not shown) into which mirror element 120 may be slid, or slipped, into such that mirror element 120 is contained within LED assembly 100 and viewable through opening 115.

In this illustrated embodiment, mirror element 120 is retained on extension arm 210, which is attached to handle 220. In one aspect of the invention, extension arm 210 may be movable with respect to handle 220 at junction 230. Junction 230 may be rigid, wherein extension arm 210 is oriented at a known angle with respect to handle 220. Alternatively, junction 230 may be flexible, wherein the orientation of extension arm 210 with respect to handle 220 may be set to a desired angle.

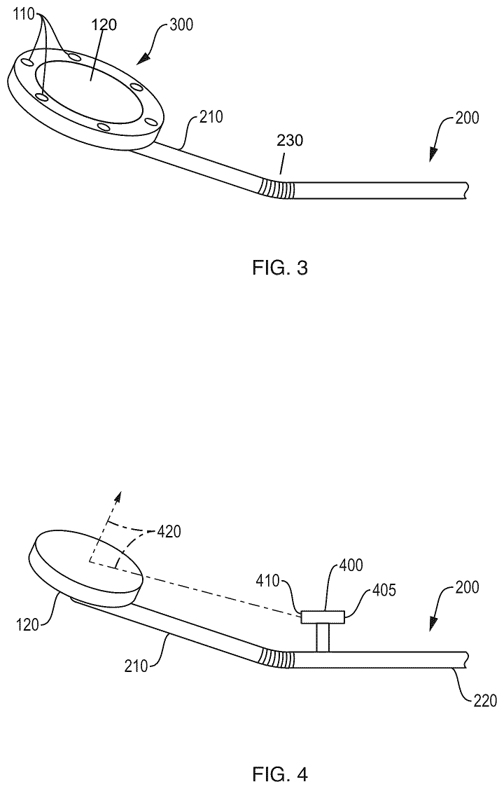

FIG. 3 illustrates a prospective side view of lighted LED assembly 100 incorporated onto mirror element 120, wherein lighted LED assembly 100 is positioned such that mirror element 120 is viewable through opening 115. In one aspect of the invention, LED assembly 100 may be removably attachable to mirror 120, as shown in FIG. 2. Alternatively, LED assembly 100 may be integral to mirror element 120.

FIG. 4 illustrates a side view of another embodiment of lighted LED assembly in accordance with the principles of the invention.

In this illustrated embodiment, lighting assembly 400 comprises housing 405 into which is positioned lighting element 410, wherein lighting assembly 400 is attachable to handle 220. Lighting elements 410, similar to lighting elements 110, emit light in one or more of the aforementioned light wavelength bands, wherein the emitted light is directed toward reflective surface 120. In accordance with the principles of this aspect of the invention, housing 405 includes electrical/electronic circuitry 140 and coiling elements 150 similar to those shown with regard to FIG. 1.

Light emitted by lighting element 410 along axis 420 is directed toward mirror 120 and re-directed by mirror 120 toward an object (not shown), such that light may be applied directly to the object. Light reflected by the not shown object may then be viewed by a practitioner through reflective surface 120.

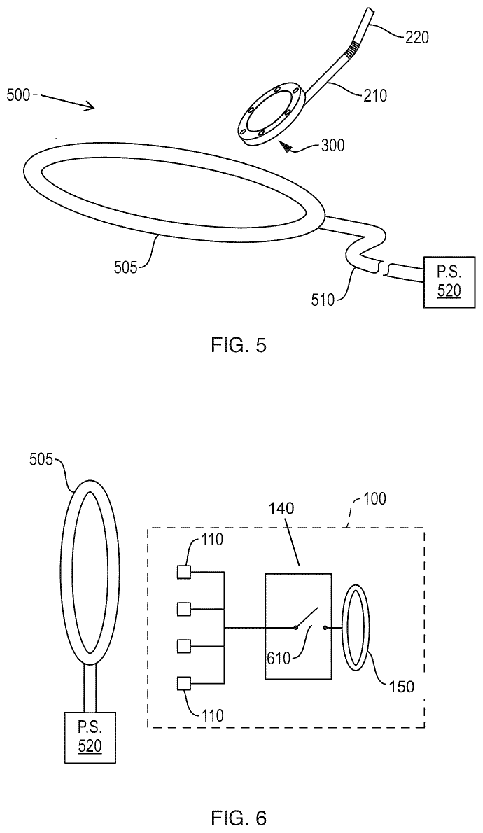

FIG. 5 illustrates a prospective view of a lighting system 500 in accordance with the principles of the invention.

In this illustrated lighting system, lighting system 500 comprises a mirror configuration similar to that shown in FIG. 3, wherein lighted LED assembly 300 is shown. Although lighting system 500 is shown with regard to lighted LED assembly 300, it would be understood that lighted LED assembly shown in FIG. 4 may be incorporated into lighting system 500 without altering the scope of the invention claimed.

In accordance with the principle of the invention, lighting system 500 further includes coil element 505, comprising a plurality of electrical coils (not shown) contained therein, which may be electrically connected to a power source 520 through wire connection 510. Although a single coil 505 is illustrated, it would be understood that the illustrated coil element 505 may comprise a plurality of coil elements 505, each of which comprises a plurality of electrical coils.

In one aspect of the invention, power source 520 may be an alternating voltage source (AC) that may be plugged into a main source of electrical energy, wherein the alternating voltage applied to coil 505 creates a fluctuating magnetic field that may be used to induce a voltage within coil 150 when device 300 is inserted within coil 505 or placed proximate to coil 505. Alternatively, power source 520 may be a direct voltage source (DC) that includes a breaker circuit that causes breaks or interruption in the substantially consistent voltage (i.e., a DC voltage) to produce a switched DC voltage that may be provided to coil(s) 505.

For example, power source 520 may comprise a battery (e.g., rechargeable), wherein the constant voltage provided by power source 520 may be periodically interrupted (switched on and off), to cause the generation of a fluctuating magnetic field within coil 505. Alternatively, power source 520 may comprise an alternating current power source that provides an alternating voltage to an alternating current/direct current (AC/DC) converter, wherein the alternating voltage is converted to a substantially constant direct current (DC) voltage that may then be applied to a switching circuit that causes interruptions in the provided substantially constant voltage. The interrupted voltage when applied to coil(s) 505 causes the generation of fluctuating magnetic field that may be used to induce a voltage within coil 150 when device 300 is inserted within, or placed proximity to, the magnetic field of coil 505.

In accordance with the principles of the invention, the alternating or fluctuating, magnetic field generated by the alternating or interrupted voltage within coil 505 induces a electrical energy within coil 150 such that the induced electrical energy may be used to cause light to be emitted by one or more of the LED elements 110.

Although not shown it would be recognized that coil 505 may be enclosed or encapsulated within a waterproof, non-toxic material (e.g., silicon) to prevent damage to coil 505 or harm a patient to whom coil 505 is placed nearby.

FIG. 6 illustrates a block diagram of a first aspect of lighting system 500, wherein coil 505 includes power source 520, which, as discussed, provides an alternating voltage or switched DC voltage to coil 505. Further illustrated is lighted LED assembly 100 comprising lighting elements 110 and coil element 150. In accordance with the principles of the invention, when coil 150 is passed within (or proximate to) the fluctuating magnetic field of coil element 505, the fluctuating magnetic field causes the induction of electrical energy in coil element 150, wherein the induced electrical energy in coil element 150 may then be provided to lighting elements 110, wherein light may be emitted by lighting elements 110.

Accordingly, when lighted LED assembly 100 is within (or proximate to) the magnetic field generated by the alternating electrical voltage within coil 505, lighting elements 110 emit light and when lighted LED assembly 100 is drawn from coil 505, the emitted light is turned off. In one aspect of the invention, electrical/electronic circuitry 140 may comprise a switch 610, wherein switch 610 controls the application of the induced voltage within coil 150 to lighting elements 110. Switch 610 may, for example, comprise a physical toggle switch, that requires switch 610 to be in a “on” position to enable voltage to be applied to lighting element 110.

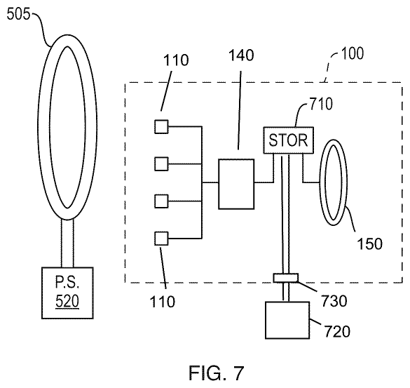

FIG. 7 illustrates a block diagram of a second aspect of lighting system 500, wherein lighted LED assembly 100 includes a storage device (e.g., a battery or super capacitor) 710 that may be used to store the induced voltage within coil 150. The stored electrical energy in storage device 710 may then be utilized to provide a substantially constant voltage to lighting elements 110.

Although not shown in FIG. 7, it would be recognized that the configuration of electrical/electronic circuitry 140 shown in FIG. 7 may include switch element 610, shown in FIG. 6, without altering the scope of the invention claimed.

In accordance with this aspect of the invention, storage device 710 may be pre-charged with a voltage that may be applied to lighting elements 110 and recharged by the voltage induced by placing coil 150 within (or proximate to) the fluctuating magnetic field caused by the alternating or interrupted voltage provided to coil 505, wherein storage device 710 is pre-charged through a serial connection (e.g., USB-c) 730 when connected to a power source 720.

In accordance with one aspect of the invention, the electrical energy (i.e., voltage, current) provided to individual lighting elements 110 may be controlled by circuitry 140 such that the light intensity emitted by different ones of lighting elements 110 may be different. Circuity 140 may comprise special purpose electronic elements (e.g., ASIC, FPGA) or dedicated electronics that allows of the selected distribution of electrical energy (e.g., voltage/current) to all or different ones of said plurality of light emitting diodes 110.

For example, limiting the current to one of lighting elements 110 to be lower than the current to another one of light elements 110, causes the emitted light of those lighting elements 110 with less current of be of a lesser intensity than the emitted light intensity of those lighting elements 110 provided the higher current.

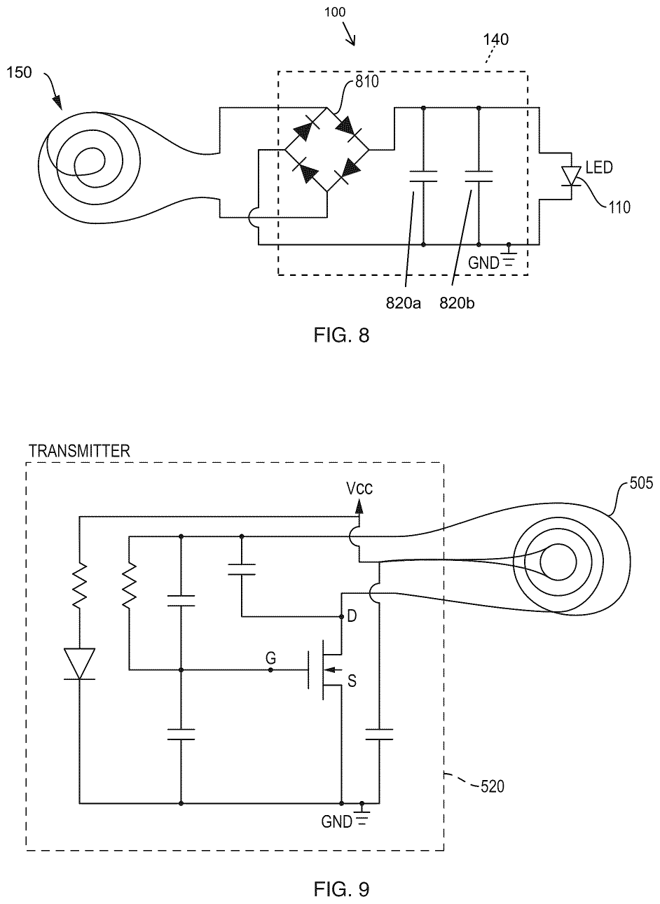

FIG. 8 illustrates an exemplary electrical circuit 140 configurated to light LEDs 110 of lighted assembly 100 in accordance with the principles of the invention.

In this illustrated exemplary configuration, an electrical energy (i.e., voltage) induced within coil 150 is converted to a substantially constant voltage (DC, direct current) by full-wave rectifier 810, wherein the substantially constant voltage may then be applied to LEDs 110. Capacitors 820a, 820b provide for reducing (i.e., smoothing) the ripple of the rectified DC voltage to provide a substantially constant voltage to LED 110. In accordance with the principles of the invention, light is emitted by LEDs 110 while coil 150 remains within (or proximity to) the fluctuating magnetic field caused by the alternating or interrupted voltage applied to coil 505 (not shown in FIG. 8). Although a full-wavelength rectifier 810 is shown, it would be known by those skilled in the art to utilize other types or configuration of electrical circuit to produce a substantially constant voltage to be applied to LEDs 110 and have been contemplated and considered within the scope of the invention claimed.

FIG. 9 illustrates an exemplary electrical circuit configuration to provide an alternating or interrupted voltage to one or more of a plurality of coils 505 through power supply 520.

In this illustrated exemplary electrical circuit configuration, a direct voltage source (not shown) provides a substantially constant voltage (i.e., Vcc)) to circuit 502, wherein the substantially constant voltage is interrupted or broken up prior to being provided to coil 505. The interrupted direct voltage causes fluctuation of a magnetic field generated within coil 505, as previously discussed, which when detected by coil 150 (see FIG. 6), causes the induction of an electrical energy within coil 150.

In summary, a lighted LED assembly is disclosed that is suitable for incorporation on to existing devices (e.g., dental mirrors) that provide light to areas viewed by the mirror element, wherein the power provided to the lighting elements 110 of the lighted LED assembly 100 is provided through a voltage induced within coil 150 within the LED assembly 100.

As used herein, the terms “comprise”, “comprising”, “includes”, “including”, “has”, “having”, or any other variation thereof, are intended to cover non-exclusive inclusions. For example, a process, method, article or apparatus that comprises a list of elements is not necessarily limited to only those elements but may include other elements not expressly listed or inherent to such process, method, article, or apparatus. In addition, unless expressly stated to the contrary, the term “of’ refers to an inclusive “or” and not to an exclusive “or.” For example, a condition A or B is satisfied by any one of the following: A is true (or present) and B is false (or not present); A is false (or not present) and B is true (or present); and both A and B are true (or present).

The terms “a” or “an” as used herein are to describe elements and components of the invention. This is done for convenience to the reader and to provide a general sense of the invention. The use of these terms in the description, herein, should be read and understood to include one or at least one. In addition, the singular also includes the plural unless indicated to the contrary. For example, reference to a composition containing “a compound” includes one or more compounds. As used in this specification and the appended claims, the term “or” is generally employed in its sense including “and/or” unless the content clearly dictates otherwise.

All numeric values are herein assumed to be modified by the term “about,” whether or not explicitly indicated. The term “about” generally refers to a range of numbers that one of skill in the art would consider equivalent to the recited value (i.e., having the same function or result). In any instances, the terms “about” may include numbers that are rounded (or lowered) to the nearest significant figure.

Although the invention disclosed herein discusses wavelengths that are produced with currently available light emitting sources (i.e., non-lasing light emitting diodes (LEDs and laser diodes (LEDs)), it would be recognized that the specific wavelengths disclosed may be changed and/or added to without altering the scope of the invention. In addition, it would be known in the art that the specific wavelengths discussed, herein, represent a band of wavelengths centered on the wavelength values presented herein to account for divergence of the wavelength generated by the LED during the generation of the light and/or the operation of the LED, wherein the wavelength value is represented as a nominal value.

Although the light emitting elements or source 110 is shown as a single element, it would be understood that the light emitting source 110 may comprise a plurality of light emitting sources (i.e., LEDs), emitting light in the same or different wavelengths, contained within a single package.

The invention has been described with reference to specific embodiments. One of ordinary skill in the art, however, appreciates that various modifications and changes can be made without departing from the scope of the invention as set forth in the claims. Accordingly, the specification is to be regarded in an illustrative manner, rather than with a restrictive view, and all such modifications are intended to be included within the scope of the invention.

Benefits, other advantages, and solutions to problems have been described above regarding specific embodiments. The benefits, advantages, and solutions to problems, and any element(s) that may cause any benefits, advantages, or solutions to occur or become more pronounced, are not to be construed as a critical, required, or an essential feature or element of any or all of the claims.

1. A lighting dental mirror system comprising:
a dental mirror; and
a LED assembly comprising:
an outer element surrounding an open center, said outer element comprising:
a plurality of light emitting diodes arranged within said outer element;
an electrical circuit configured to:
provide a voltage to at least one of said plurality of light emitting diodes; and
a coil positioned within said outer element, said coil configured to:
cause to have generated an electrical energy by an induction process when said mirror system is proximal to a varying magnetic field; and
provide said electrical energy to said electrical circuit, wherein said electrical circuit comprising:
a rectifier circuit configured to:
convert said electrical energy provided by said coil to said voltage applied to said at least one of said plurality of light emitting diodes.

2. The lighted dental mirror system of claim 1, wherein said LED assembly is removably attachable to said dental mirror.

3. The lighted dental mirror system of claim 2, wherein said attachment of said LED assembly to said dental mirror is one of: a snap-fit connection, a screw tread connection, a bayonet connection, slip-in and a slide-on connection.

4. The lighted dental mirror system of claim 1, wherein said outer element comprises:
an optical clear material.

5. The lighted dental mirror system of claim 1, wherein electrical circuit is configured to:
apply said voltage to selected one ones of said plurality of light emitting diodes differently than to other selected ones of said plurality of light emitting diodes.

6. The lighted dental mirror system of claim 1, wherein said light emitting diodes are configured to emit light in at least one of: a white wavelength range and a blue wavelength range.

7. The lighted dental mirror system of claim 1 comprising:
a storage medium, wherein said voltage is stored within said storage medium.

8. The lighted dental mirror system of claim 7, wherein said stored voltage is applied to at least one of said plurality of light emitting diodes.

9. A dental mirror system comprising:
an inductive source; and
a lighted dental mirror, wherein said inductive source comprises:
a first coil comprising a plurality of coils arranged about an opening; and
a power supply configured to generate a fluctuating magnetic field withing said first coil; and
said lighted dental mirror configured to be positionable within said fluctuating magnetic field, said light dental mirror comprises:
a dental mirror assembly comprising:
a handle: and
a dental mirror extending from said handle; and
a lighting assembly attached to said dental mirror, wherein said lighting assembly comprises:
a plurality of light emitters;
an electrical circuit configured to:
apply a voltage to at least one of said light emitters; and
a second coil configured to:
generate an electrical energy when placed within or proximate to said fluctuating magnetic field associated with said first coil, wherein said electrical circuit comprises:
a rectifier circuit configured to:
preceive said electrical energy; and
convert said electrical energy provided by said second coil to said voltage applied to said at least one of said plurality of light emitters.

10. A dental mirror system of claim 9, wherein said attachment of said light assembly to said dental mirror is one of: a snap-fit connection, a screw tread connection, a bayonet connection, slip-in and a slide-on connection.

11. The lighted dental mirror system of claim 9, wherein said lighting assembly comprises:
an optical clear material.

12. The lighted dental mirror system of claim 9, wherein electrical circuit is configured to:
apply said voltage to selected one ones of said plurality of light emitters differently than to other selected ones of said plurality of light emitters.

13. The lighted dental mirror system of claim 9, wherein said light emitters are configured to emit light in at least one of: a white wavelength range and a blue wavelength range.

14. The lighted dental mirror system of claim 9 comprising:
a storage medium, wherein said voltage is stored within said storage medium.

15. The lighted dental mirror system of claim 14, wherein said stored voltage is applied to at least one of said plurality of light emitters.

16. The lighted dental mirror system of claim 9, wherein said lighting assembly is one: removably attachable to and integral to said dental mirror.
